(19) United States
(12) Patent Application Publication
(10) Pub. No.: US 2012/0258170 A1
(43) Pub. Date: Oct. 11, 2012

(54) PHARMACEUTICAL CO-CRYSTALS OF QUERCETIN

(75) Inventors: Anil Kumar KRUTHIVENTI, Hyderabad (IN); Iqbal Javed, Hyderabad (IN); Satyanarayana Reddy Jaggavarapu, Hyderabad (IN); RaviKumar Nagalapalli, Hyderabad (IN); Ganesh Saraswatula Viswanadha, Hyderabad (IN); Solomon Kamalakaran Anand, Hyderabad (IN)

(73) Assignee: Nutracryst Therapeutics Private Limited, Noida (IN)

(21) Appl. No.: 13/423,728

(22) Filed: Mar. 19, 2012

Related U.S. Application Data
(63) Continuation-in-part of application No. 13/382,704, filed on Jan. 6, 2012.

(30) Foreign Application Priority Data
May 20, 2009 (IN) 1154/CHE/2009

(51) Int. Cl. A61K 31/353; C07D 487/04; A61K 31/4985; C07D 207/16; A61K 31/40; C07F 5/02; A61K 31/69; C07D 209/52; A61K 31/403; C07D 473/04; A61K 31/522; C07D 471/04; A61K 31/519; C07D 401/04; A61K 31/506; A61K 31/55; A61K 31/4965; A61K 31/4015; A61K 31/7036; A61K 31/4439; A61K 31/44; A61K 9/20; A61P 27/12; A61P 3/00; A61P 9/12; A61P 3/10; A61P 3/06; A61P 3/04; C07D 311/32

(52) U.S. Cl. 424/464; 549/400; 514/456; 544/350; 514/249; 548/528; 514/423; 548/405; 514/64; 548/452; 514/412; 544/268; 514/263.21; 544/279; 514/264.1; 544/312; 514/274; 514/217.11; 514/255.06; 514/424; 514/42; 514/342; 514/348

(57) ABSTRACT

A co-crystal composition comprised of Quercetin and at least one antidiabetic agent acts as a combination drug having unique physical properties and biological activity, which differ from both Quercetin in pure form and the at least one antidiabetic agent in pure form. The co-crystal composition may comprise quercetin and metformin. The co-crystals of quercetin and metformin may be prepared by grinding the compounds, and used in pharmaceutical compositions comprising these co-crystals. Co-crystal compositions of quercetin and Metformin may be used in combination with other anti-diabetic agents, including DPP-IV inhibitors.

### CROSS-REFERENCE TO RELATED APPLICATIONS

This application is a continuation-in-part of parent U.S. application Ser. No. 13/382,704 filed on Jan. 6, 2012. The entire disclosure of the prior application is hereby incorporated by reference herein in its entirety.

### TECHNICAL FIELD

This disclosure relates to the field of pharmaceutical co-crystals of Quercetin. More particularly, the present disclosure relates to synergistic pharmaceutical co-crystals comprising Quercetin and an anti diabetic agent(s) as combination drug that have unique physical properties and biological activity which differ from the active agent in pure form, to process for preparation of the same and also relates to pharmaceutical compositions comprising these synergistic co-crystals.

### BACKGROUND AND PRIOR ART

Even though co-crystals were known as early as 19^{th} century, the pharmaceutical industry has recognized the potential for their applications only recently. Pharmaceutical co-crystals are crystalline molecular complexes containing therapeutic molecules. These co-crystals represent emerging class of pharmaceutical materials offering the prospects of optimized physical properties.

The application of co-crystallization can further be extended to neutral molecules, amorphous compounds, to improve their physio-chemical properties. Further, these co-crystals have utility in imparting desirable physical properties and stability, which are otherwise not achievable for the pure active agent or in combination as a simple formulation using the excipients incorporated with the active agent.

Quercetin is a plant-derived flavonoid, specifically a flavonol, used as a nutritional supplement.

The American Cancer Society says that Quercetin has been promoted as being effective against a wide variety of diseases, including cancer. As high dietary intake of fruits and vegetables is associated with reduction in cancer, and therefore scientists suspect Quercetin may be partly responsible. Quercetin is the aglycone form of a number of other flavonoid glycosides, such as rutin and quercitrin, found in citrus fruit, buckwheat and onions. Quercetin forms the glycosides quercitrin and rutin together with rhamnose and rutinose, respectively. Quercetin is classified as IARC group 3 (no evidence of carcinogenicity in humans).

Further, Quercetin is an Anti-tumor agent; induces apoptosis and inhibits synthesis of heat shock proteins. Quercetin, one of the most widely distributed flavonoids in the plant kingdom inhibits many enzyme systems including tyrosine protein kinase, phospholipase A₂, phosphodiesterases, mitochondrial ATPase, PI 3-kinase and protein kinase C; can also activate Ca²⁺ and K⁺ channels [Merck Index].

Quercetin, one of the most widely distributed flavonoids in the plant kingdom, inhibits various enzymes. The inhibitory effect of quercetin on the angiotensin-converting enzyme activity through the cardiovascular response to bradykinin and angiotensin I has been examined. Quercetin pretreatment (88.7 μmol/kg p.o., 45 min; 14.7 μmol/kg i.v., 5 min) significantly potentiated the hypotensive effect of bradykinin (10 nmol/kg i.v.). This association was significantly attenuated by an antagonist of the B2 receptor. In addition, the hypertensive response to angiotensin I (0.1 nmol/kg iv) was significantly reduced by Quercetin pretreatment using the same parameters as before. These results suggest an inhibitory effect of Quercetin on the angiotensin-converting enzyme activity, similar to that of captopril. Quercetin was equally effective when given orally or intravenously.

Pre-incubation of cells with Quercetin followed by cisplatin treatment appeared to be the most effective and was correlated with strong activation of caspase-3 and inhibition of both heat shock proteins (Hsp72) and multi-drug resistance proteins (MRP) levels. The results indicate that Quercetin pretreatment sensitizes HeLa cells to cisplatin-induced apoptosis in HeLa cells. Quercetin and rutin may also be useful in the treatment of IAR and LAR in asthma via inhibition of histamine release, PLA2, and EPO, and reduced recruitment of neutrophils and eosinophils into the lung.

Glucose-induced changes in HepG2 cells in AMP-activated protein kinase (AMPK) activity may be mediated by SIRT1, an NAD⁺-dependent histone/protein deacetylase that has been linked to the increase in longevity caused by caloric restriction. Incubation with 25 vs. 5 mM glucose for 6 h concurrently diminished the phosphorylation of AMPK (Thr 172) and ACC (Ser 79), increased lactate release, and decreased the abundance and activity of SIRT1. In contrast, incubation with pyruvate (0.1 and 1 mM) for 2 h increased AMPK phosphorylation and SIRT1 abundance and activity. The putative SIRT1 activators resveratrol and Quercetin also increased AMPK phosphorylation. None of the tested compounds (low or high glucose, pyruvate, and resveratrol) significantly altered the AMP/ATP ratio. Collectively, these findings raise the possibility that glucose-induced changes in AMPK are linked to alterations in SIRT1 abundance and activity and possibly cellular redox state.

Recent findings demonstrate that Quercetin bioavailability has been underestimated in the past and can be improved by food matrix components or particular delivery forms. Among the biological effects of particular relevance, the antihypertensive effects of Quercetin in humans and the improvement of endothelial function should be emphasized. Together with its antithrombotic and anti-inflammatory effects, the latter mainly mediated through the inhibition of cytokines and nitric oxide; Quercetin is a candidate for preventing obesity-related diseases. Most exiting are the findings that Quercetin enhances physical power by yet unclear mechanisms. The anti-infectious and immunomodulatory activities of Quercetin might be related to this effect.

Quercetin increased mRNA expression of PGC-1alpha and SIRT1 (P<0.05), mtDNA (P<0.05) and cytochrome C concentration (P<0.05). These changes in mitochondrial capacity were associated with an increase in both maximal endurance capacity (P<0.05) and voluntary wheel running activity (P<0.05). These benefits of querectin on fitness without exercise training may have important implications for enhancement of athletic and military performance and may also extend to prevention and/or treatment of chronic diseases.

There is ample patented literature available on Quercetin. WO/2008/011364 discloses a composition containing Quercetin, vitamin B3, vitamin C, and folic acid. Also disclosed is a method of using the composition for enhancing physical or mental performance or treating various diseases or disorders.

US 20080031940 describes a composition includes 10-50 wt. % Quercetin along with papain; calcium salt; zinc salt; bee pollen; pumpkinseed; bromelain; and saw palmetto; wherein the composition is a sustained release composition in tablet or capsule form suitable for oral administration to a human. Methods of making and using the composition are provided.

Method for preventing or treating elevated blood lipid level-related diseases by administering rutin and Quercetin is disclosed in U.S. Pat. No. 6,509,372.

WO/2002/076473 describes Quercetin, its preparation and the medicinal composition containing the same and their application for preventing or treating diseases related to 5HT14 receptor or neure damage, including preventing or treating Alzeheimer's disease, drug or alcohol dependence, sleep disorders or panic state, delaying senility or improving memory function and preventing or treating neure damage caused by brain injury.

However, the prior art fails to teach or suggest pharmaceutical co-crystals of Quercetin, useful in effective treatment of disease conditions. Pharmaceutical co-crystals are crystalline molecular complexes containing therapeutic molecules. These co-crystals represent an emerging class of pharmaceutical materials offering the prospects of optimized physical as well as therapeutic properties.

Therefore, various embodiments disclosed herein relate to synergistic pharmaceutical co-crystals of Quercetin using different active molecules as combination drugs selected from anti-diabetic agents.

### SUMMARY

In accordance with the above objective, the present disclosure describes synergistic pharmaceutical co-crystals of Quercetin and anti-diabetic agents selected from biguanide group like metformin; sulfonylurea group like tolazamide, glipizide, glimepiride; Alpha-glucosidase inhibitor such as acarbose); a second generation α-glucosidase inhibitor, miglitol; members of the thiazolidinedione class such as rosiglitazone, pioglitazone; and DPP-IV inhibitors selected from sitagliptin, vildagliptin, dutogliptin, saxagliptin, linagliptin, gemigliptin and alogliptin. These synergistic pharmaceutical co-crystals act as a combination drug for metabolic syndrome associated with Hypertension, diabetic conditions inclusive of obesity and hyperlipidemia.

Accordingly in one embodiment, the present disclosure provides the co-crystals of Quercetin which have been prepared using the above antidiabetic agents. These co-crystals showed higher solubility, dissolution rates and also found to be stable under accelerated conditions and thus suitable in preparing pharmaceutical compositions.

Accordingly in one aspect of the present disclosure, pharmaceutical co-crystal specifically comprises Quercetin dehydrate and Metformin as a combination drug. The co-crystal formed is further analyzed and characterized using PXRD, IR and Mass spectra. The present inventors have surprisingly noted that the melting point of the Quercetin dihydrate-Metformin co-crystal is much lower than Quercetin and higher than Metformin. Further, the solubility of Quercetin is also substantially improved when it is delivered as a co-crystal with a water soluble drug like Metformin.

Various embodiments of the present disclosure relate to a co-crystal of quercetin and metformin in physical combination with an anti-diabetic agent selected from the group consisting of DPP-IV inhibitor. In various embodiments, the present disclosure provides a co-crystal of quercetin and metformin in physical combination with at least one DPP-IV inhibitor selected from the group consisting of sitagliptin, vildagliptin, dutogliptin, saxagliptin, linagliptin, gemigliptin, alogliptin and mixture thereof. In some embodiments, the present disclosure provides a co-crystal of quercetin and metformin in physical combination with sitagliptin.

In some embodiments, the present disclosure describes a process for preparing Quercetin dihydrate-Metformin co-crystals by hand grinding. In other embodiments, the present disclosure describes a process for preparing Quercetin dihydrate-Metformin co-crystals by a method of melting the two drugs or by a solvent drop method using suitable solvents. The process for preparing Quercetin dihydrate-Metformin co-crystals by hand grinding, by melting, or by a solvent drop method may be followed by crystallization, if necessary.

In yet another aspect, the disclosure provides process for preparation of pharmaceutical co-crystals of Quercetin-antidiabetic agent wherein said process comprises providing Quercetin and an antidiabetic agent; isolating said co-crystal and incorporating it into pharmaceutical composition along with one or more suitable pharmaceutical carriers/excipients. The co-crystals disclosed herein, optionally in combination with excipients, can be formulated into compositions and dosage forms according to methods known in the art.

In yet another aspect, the current disclosure describes a method for treating metabolic syndrome associated with Hypertension, diabetic conditions inclusive of obesity and hyperlipidemia, which method comprises administering an effective amount of co-crystals of Quercetin and an antidiabetic agent to the subject suffering from said disorder. The subject mentioned herein is human.

In yet another aspect, the current disclosure describes use of the co-crystals of Quercetin and an antidiabetic agent in preparing a medicament intended to treat metabolic syndrome associated with Hypertension, diabetic conditions inclusive of obesity and hyperlipidemia.

### DESCRIPTION OF DRAWINGS

FIG. 1 shows the IR spectra of Metformin+Quercetin co-crystals

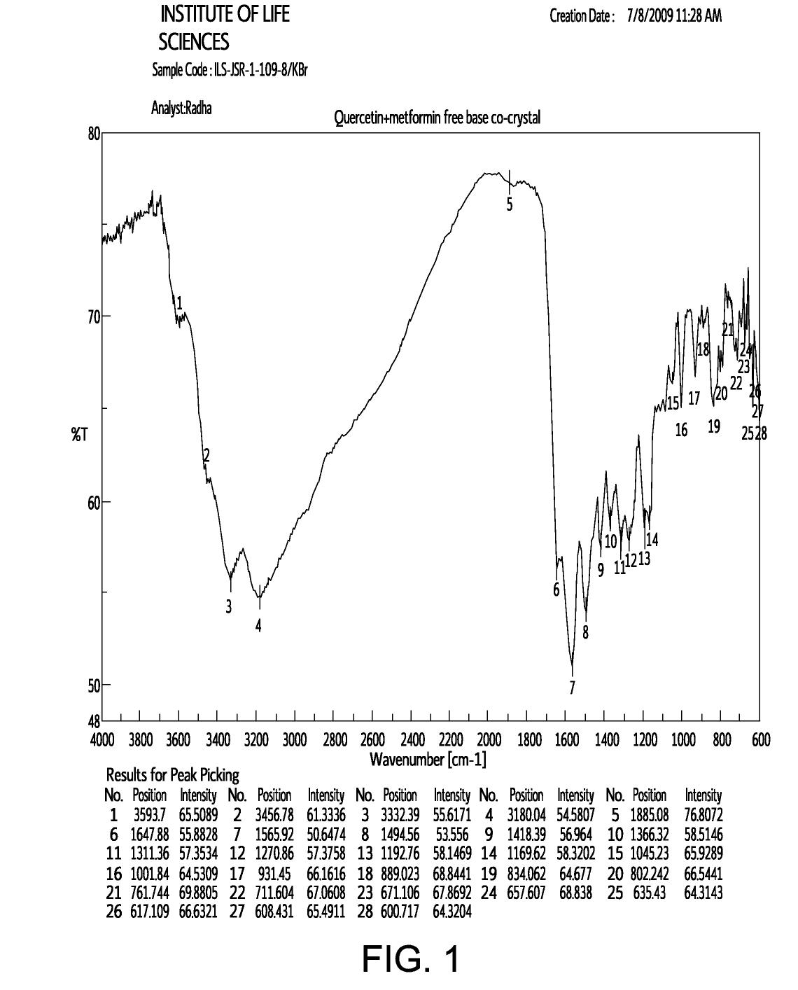

FIG. 2 shows PXRD of Metformin+Quercetin co-crystals

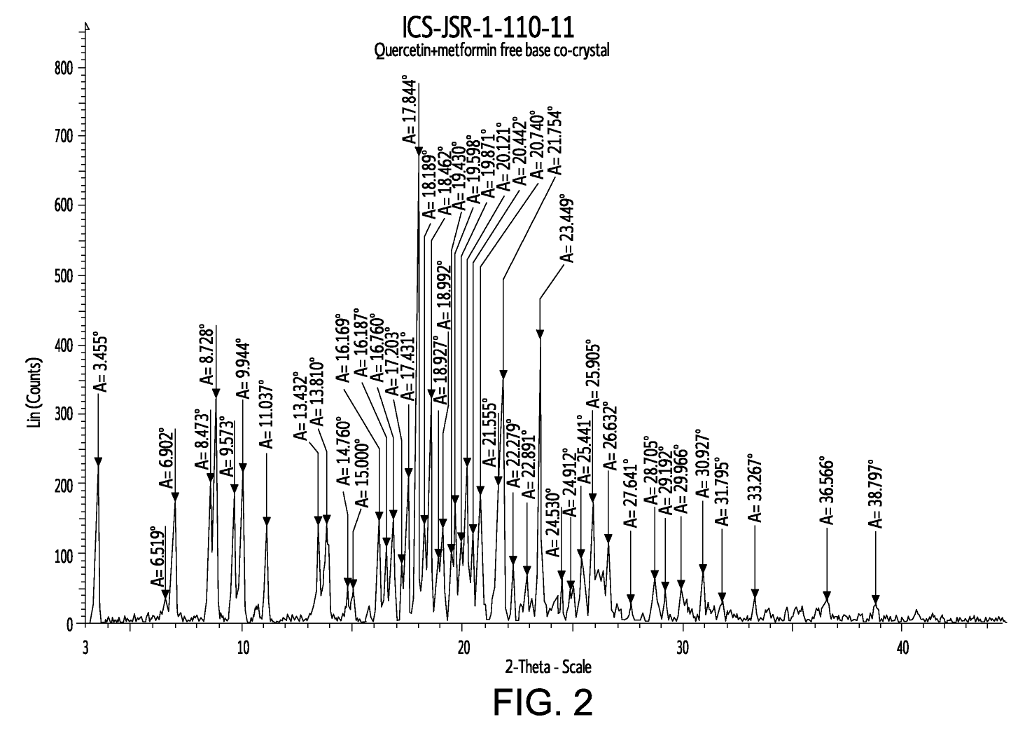

FIG. 3 shows DSC of Metformin+Quercetin co-crystals

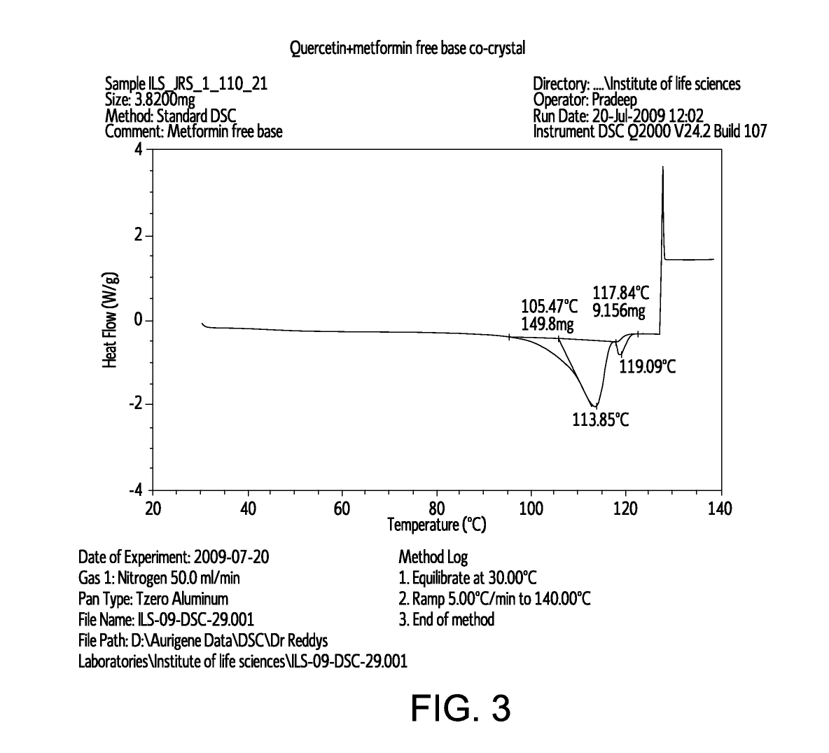

FIG. 4 shows NMR (PPM) of Metformin+Quercetin co-crystals

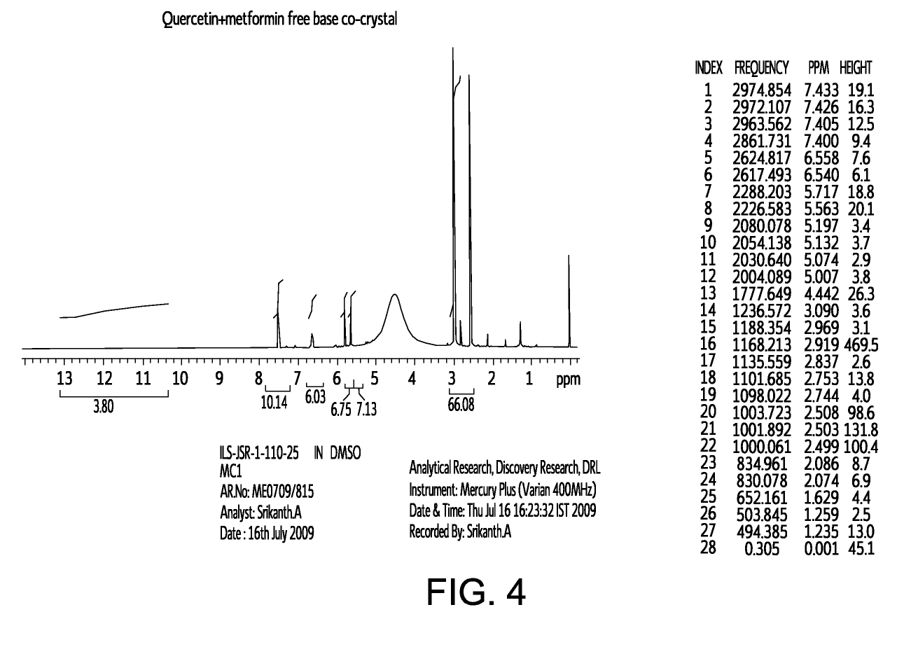

FIG. 5 shows TGA of Metformin+Quercetin co-crystal

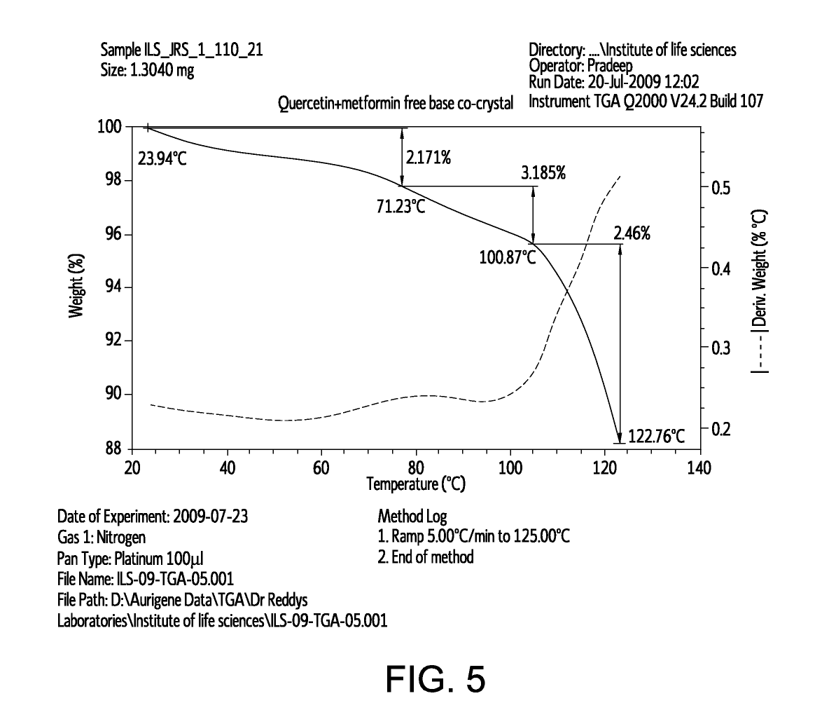

FIG. 6 shows the IR spectra of Metformin HCl+Quercetin co-crystals

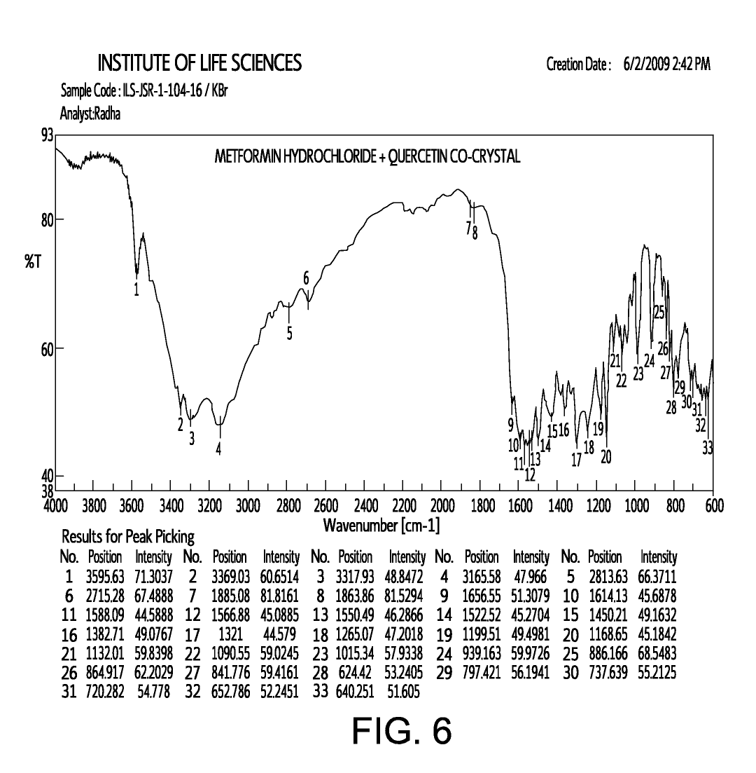

FIG. 7 shows PXRD of Metformin HCl+Quercetin co-crystals

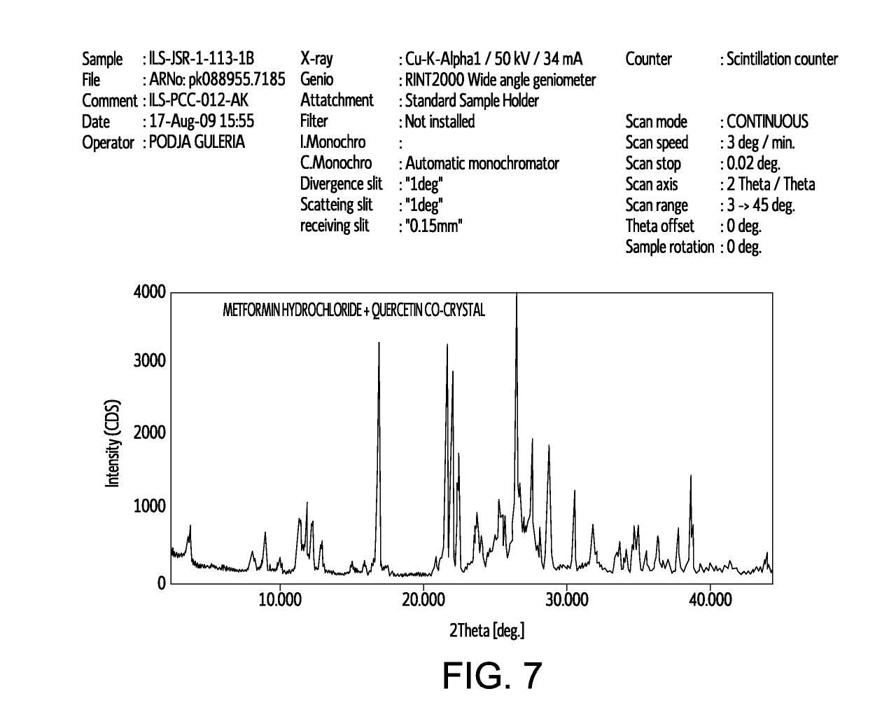

FIG. 8 shows DSC of Metformin HCl+Quercetin co-crystals

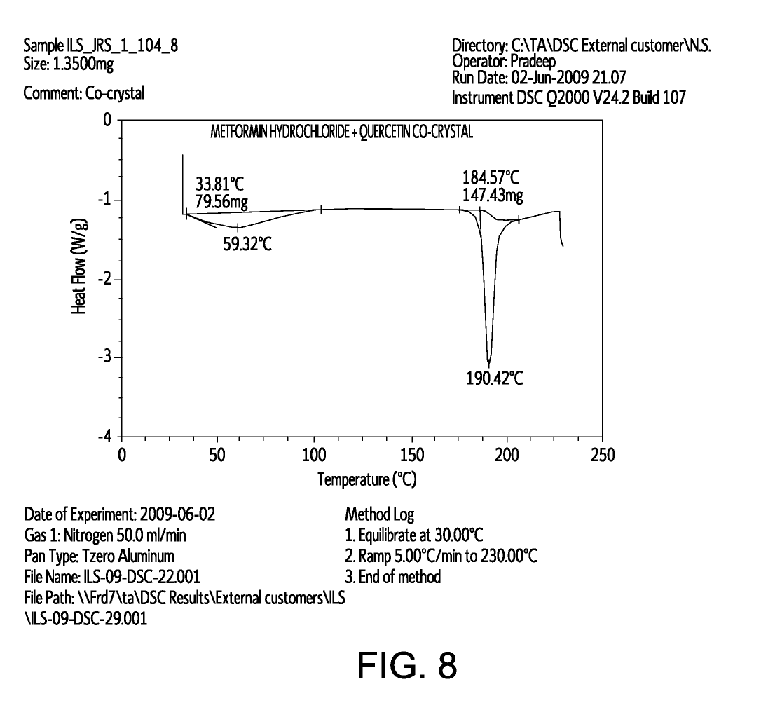

FIG. 9 shows TGA of Metformin HCl+Quercetin co-crystal

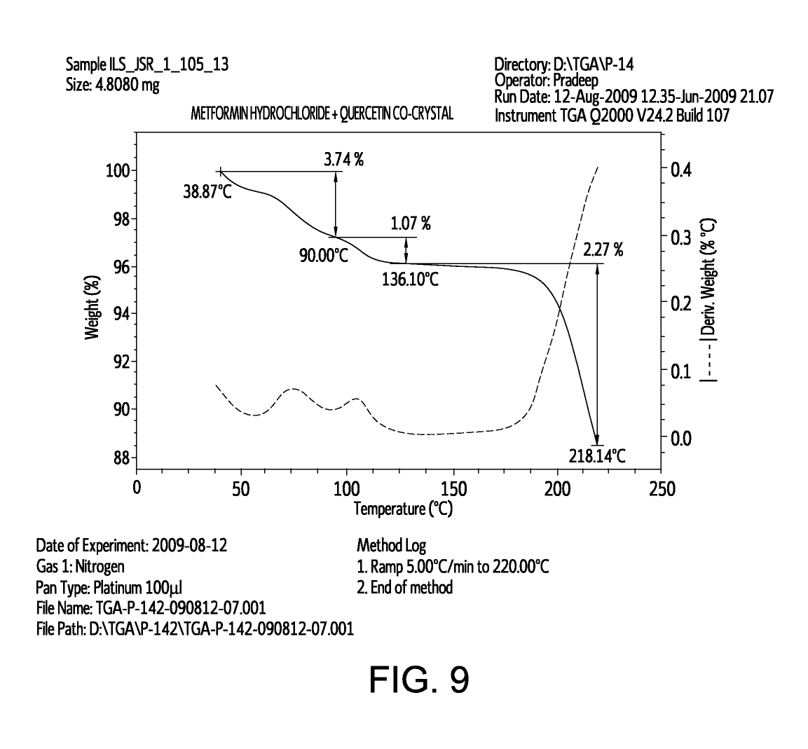

FIG. 10 shows the Experimental Design for experiments on male db/db mice

### ABBREVIATION

ILB-MCO-0904Q=Quercetin dihydrate

ILB-MCO00905Q=Quercetin dihydrate+Metformin free base co-crystal (1:2)

ILB-MCO-0906Q=Metformin hydrochloride

### DETAILED DESCRIPTION

The invention will now be described in detail in connection with certain preferred and optional embodiments, so that various aspects thereof may be more fully understood and appreciated.

Metformin is an oral anti-diabetic drug from the biguanide class. It is the first-line drug for the treatment of type 2 diabetes, particularly in overweight and obese people and those with normal kidney function, and evidence suggests it may be the best choice for people with heart failure. It is also used in the treatment of polycystic ovary syndrome. Metformin is the only anti-diabetic drug that has been proven to protect against the cardiovascular complications of diabetes. Metformin also modestly reduces LDL and triglyceride levels.

There is ample literature available on Metformin hydrochloride and the pharmaceutical compositions comprising the same for the treatment of type II diabetes.

In this application, the inventors have achieved the pharmaceutical co-crystals of Quercetin using numerous antidiabetic agents as combination drug, which works synergistically against metabolic syndrome associated with Hypertension, diabetic conditions inclusive of obesity and hyperlipidemia, thus enhancing the efficacy of the combination even in lower doses.

Quercetin demonstrates significant anti-inflammatory activity via a number of actions including inhibition of mast cell and basophil degranulation, neutrophil and monocyte lysosomal secretion, prostaglandin formation (particularly leukotrienes), and lipid peroxidation. A membrane stabilizing effect, is known to be part of the anti-inflammatory effect. Quercetin has also been shown to inhibit the enzyme hyaluronidase (responsible for the breakdown of collagen matrix of connective tissue and ground substance). Leukotrienes promote inflammation by causing vaso-constriction, which can then lead to vascular permeability and broncho constriction.

Quercetin's anti-inflammatory activity appears to be due to its antioxidant and inhibitory effects on inflammation-producing enzymes (cyclooxygenase, lipoxygenase) and the subsequent inhibition of inflammatory mediators, including leukotrienes and prostaglandins Inhibition of histamine release by mast cells and basophils also contributes to quercetin's anti-inflammatory activity.

Aldose reductase, the enzyme which catalyzes the conversion of glucose to sorbitol, is especially important in the eye, and plays a part in the formation of diabetic cataracts and retinopathy. Quercetin is a strong inhibitor of human lens aldose reductase.

Accordingly, the disclosure describes synergistic pharmaceutical compositions comprising the co-crystals of Quercetin and anti-diabetic agents. The antidiabetic agent is selected from biguanide group like metformin; sulfonylurea group like tolazamide, glipizide, glimepiride; Alpha-glucosidase inhibitors such as acarbose; a second generation a-glucosidase inhibitor, miglitol; members of the thiazolidinedione class such as rosiglitazone, pioglitazone; DPP-IV inhibitors selected from sitagliptin, vildagliptin, dutogliptin, saxagliptin, linagliptin, gemigliptin and alogliptin; and members of the thiazolidinedione class such as rosiglitazone or pioglitazone. The co-crystals of Quercetin and anti-diabetic agents act as a combination drug for metabolic syndrome associated with Hypertension, diabetic conditions inclusive of obesity and hyperlipidemia. Quercetin can be used in the form of its hydrates, its salts, or its polymorphs.

The co-crystal composition consisting of Quercetin and metformin, when used in a physical combination with DPP-IV inhibitors as anti-diabetic agent, reduces the required dose of the DPP-IV inhibitors. Since, as seen from the table below, neither sitagliptin (DPP IV inhibitor), nor metformin have proper plasma protein binding, cocrystal of metformin and quercetin in combination with DPP-IV inhibitors (sitagliptin) prove better bioavailable medication.

| S. | | Bioavail- | Half | Plasma Protein | |
|---|---|---|---|---|---|
| NO: | Compound | ability | Life | Interaction | Side Effects |
| 1 | Sitagliptin | 87% | 8-14 | 38% | Nausea |
| | | | hours | | |
| 2 | Metformin | 46% | 6.2 | negligibly | Lactic acidosis, |
| | alone | | hours | bound | diarrhea, |
| | | | | | flatulence |

Further, since, metformin is absorbed paracellularly rather than transcellular absorption and since, quercetin is absorbed transcellular, it is observed by the present inventor that in co-crystal of quercetin and metformin, metformin's mode of absorption is altered significantly thus leading to significant increased bioavailability and other pharmacokinetic properties.

Accordingly in one embodiment, the present disclosure describes co-crystals of Quercetin or its dehydrate; and Metformin, pharmaceutical metformin salts, and metformin polymorphs. Co-crystals of Quercetin and Metformin act as a combination drug for metabolic syndrome associated with Hypertension, diabetic conditions inclusive of obesity and hyperlipidemia. Also, said co-crystal compositions of Quercetin and Metformin are used for treating diabetic cataracts, due to the ability of quercetin to inhibit lens aldose reductase. These co-crystals showed high solubility and high dissolution rates, and were also found to be stable under accelerated conditions and thus suitable in preparing pharmaceutical compositions.

In another embodiment, the present disclosure describes synergistic pharmaceutical compositions comprising the co-crystals of Quercetin and metformin in physical combination with other anti-diabetic agents selected from sulfonylurea group like tolazamide, glipizide, glimepiride; Alpha-glucosidase inhibitors such as acarbose; a second generation a-glucosidase inhibitor, miglitol; members of the thiazolidinedione class such as rosiglitazone, pioglitazone; and DPP-IV inhibitors selected from sitagliptin, vildagliptin, dutogliptin, saxagliptin, linagliptin, gemigliptin and alogliptin; members of the thiazolidinedione class such as rosiglitazone, pioglitazone. Such co-crystals of Quercetin and metformin in physical combination with other anti-diabetic agents act as a combination drug for metabolic syndrome associated with Hypertension and diabetic conditions inclusive of obesity and hyperlipidemia.

In another embodiment, the present disclosure describes synergistic pharmaceutical compositions comprising the co-crystals of Quercetin and metformin in physical combination with a DPP-IV inhibitor selected from the group consisting of sitagliptin, vildagliptin, dutogliptin, saxagliptin, linagliptin, gemigliptin and alogliptin. In certain embodiments, the present disclosure describes synergistic pharmaceutical compositions comprising the co-crystals of Quercetin and metformin in physical combination with sitagliptin.

In another embodiment, the disclosure describes a process for preparation of co-crystal of Quercetin dihydrate-Metformin in the ratio of about 1:1 to about 1:3; by hand grinding. Optionally, the co-crystals described herein can be prepared by melting method, solvent drop method using suitable solvents, followed by crystallization, if necessary.

In one of the embodiments, the process for preparation of Quercetin dihydrate and Metformin free base (1:2) co-crystal comprises of ground neating the heated Quercetin dihydrate with Metformin free base for 3 minutes using a mortar and pestle. The Metformin free base is prepared by dissolving 1:1 molar ratio of Metformin hydrochloride and sodium hydroxide in 2-propanol. Further, the process for preparation of Quercetin dihydrate and Metformin hydrochloride (1:2) co-crystal comprises neat (solvent less) grinding of Metformin hydrochloride and Quercetin dihydrate for 3 minutes to make it a homogeneous mixture. The resultant product so obtained was subjected to analytical studies.

X-Ray Powder Diffractometry:

Powder data were collected on PANalytical, X'Pert PRO X-ray powder diffractometer using a parallel beam of monochromated Cu-K_{α} radiation (λ=1.5418 Å) and an X'Celerator detector at 45 kV voltage and 40 mA Current. Diffraction patterns were collected over the 2θ range 3-45°.

Further, these co-crystals were characterized by thermal analysis. The inventors have surprisingly noted that the melting point of the Metformin-Quercetin dihydrate co-crystal formed is much lower than the Quercetin and higher than Metformin.

1. The Physical Characteristics of Quercetin Dihydrate-Metformin Hydrochloride (1:2) Co-Crystals are as Tabulated Below:

| Quercetin | | | | |
|---|---|---|---|---|
| dihydrate and | | | | |
| Metformin HCl | Method | IR | M.P | PXRD |
| 1:2 | Neat(solventless) | slight | 234-241° C. | Change is |
| | Grinding. | change | | observed. |

| Characterization | | Metformin | Quercetin dihydrate and |
|---|---|---|---|
| Method | Quercetin | HCl | Metformin HCl co-crystal |
| MELTING POINT(° C.) | 300° C. | 222° C. to 226° C. | 234-241° C. |
| IR DATA: | 3590 cm⁻¹, 3409 cm⁻¹, | 3370 cm⁻¹, 3298 cm⁻¹, | 3391 cm⁻¹, 3368 cm⁻¹, |
| | 3319 cm⁻¹, 1664 cm⁻¹, | 3168 cm⁻¹, 2814 cm⁻¹, | 3292 cm⁻¹, 3162 cm⁻¹, |
| | 1613 cm⁻¹ | 2698 cm⁻¹, 1630 cm⁻ | 2882 cm⁻¹, 1653 cm⁻¹, |
| | 1561 cm⁻¹, 1522 cm⁻¹, | 1575 cm⁻¹, 1482 cm⁻¹, | 1617 cm⁻¹, 1588 cm⁻¹, |
| | 1321 cm⁻¹, 1169 cm⁻¹, | 1065 cm⁻¹ | 1509 cm^{-1,} 1362 cm⁻¹, |
| | 1015 cm⁻¹ | | 1320 cm⁻¹ 1065 cm⁻¹ |
| PXRD DATA | 10.500, 11.30, 11.46, | 12.238, 12.394, 12.905, | 4.489, 9.719, 12.65, |
| (2Theta): | 11.86, 12.16 | 17.855, 18.522, 21.831, | 13.003, 17.575, 22.26, |
| | 14.42, 15.56, 27.14 | 22.531, 23.421, 24.713, | 23.1552 5.98, 28.20, |
| | | 26.604, 29.681, 32.7513 | 29.400 |
| DSC | Sharp peak observed | Sharp peak observed | Broad peak observed |
| | at 320° C. | at 227° C. | at 190° C. |

2. The Physical Characteristics of Quercetin Dihydrate-Metformin (1:2) Co-Crystals are as Tabulated Below:

| CHARACTERISATION | Quercetin | | Quercetin dihydrate and |
|---|---|---|---|
| METHODS | (150° C. heated) | Metformin | Metformin co-crystal |
| IR(cm⁻¹) | 3312 cm⁻¹, 1668 cm⁻¹, | 3368 cm⁻¹, 3297 cm⁻¹, | 3456 cm⁻¹, 3332 cm⁻¹, |
| | 1617 cm⁻¹, 1560 cm⁻¹, | 3179 cm⁻¹, 1635 cm⁻¹, | 3180 cm⁻¹, 1647 cm⁻¹, |
| | 1513 cm⁻¹ 1357 cm⁻¹, | 1619 cm⁻¹, 1059 cm⁻¹ | 1565 cm⁻¹, 1494 cm⁻¹, |
| | 1163 cm⁻¹, 1093 cm⁻¹ | | 1418 cm⁻¹, 1311 cm⁻¹, |
| | | | 1192 cm⁻¹, 1045 cm⁻¹ |
| PXRD(2 THETA) | 4.88, 8.69, 10.89, | 12.13, 12.74, 15.74, | 3.45, 6.90, 8.47, 8.72, |
| | 11.29, 12.55, 14.43, | 16.29, 17.78, 18.24, | 9.57, 9.94, 13.43, |
| | 26.95, 27.55 | 22.73, 23.16, 24.41, | 13.81, 16.16, 16.76, |
| | | 25.89, 27.73, 29.20 | 17.43, 17.84, 18.46, |
| | | | 19.58, 20.74, 21.75, |
| | | | 23.44, 25.90 |
| MELTING POINT(° C.) | 320° C. | 101-105° C. | 120-129° C. |
| DSC | Sharp peak observed | Broad peak observed | Broad peak observed |
| | at 320° C. | at 111° C. | at 119° C. |
| NMR(PPM) | δ12.492(1H, S), | δ2.931(6H, S), | δ 2.931(6H, S) |
| | δ10.775(1H, S), | δ 6.743(4H, S) | δ 7.67(1H, d), |
| | δ 9.58(1H, S), | δ7.213(2H, S) | δ 7.68(1H, d), |
| | δ9.35(1H, S) | | δ 6.89(1H, d) |
| | δ 9.29(1H, S), | | δ 6.87(1H, S), |
| | δ7.67(1H, d), | | δ 6.18(1H, S) |
| | δ 7.68(1H, d), | | |
| | δ 6.89(1H, d) | | |
| | δ 6.87(1H, S), | | |
| | δ 6.18(1H, S | | |
| TLC in 50% EtOAc + | 0.6 | 0.0 | 0.0 & 0.6 |
| Hexane R_{f} | | (doesn't move | |
| | | in TLC) | |
| | | Presence indicated | |
| | | by ninhydrin | |
| TGA | No weight loss | Weight loss | At 105.6° C. two water |
| | observed below | observed at | molecule weight loss |
| | 300° C. | 147° C. is around | was observed and at |
| | | 46 mass | 248.14° C. & 144.9 |
| | | | weight loss observed |

Dissolution and Solubility:

The compounds disclosed herein are finely ground and passed through standard mesh filter and particles with 75-180 micron, and are tested for their solubility and dissolution profile.

According to the present disclosure, the pharmaceutical co-crystals of Quercetin dihydrate and Metformin have showed higher dissolution rates when compared to the parent molecules and were also found to be stable under accelerated conditions. Further, the solubility of the Quercetin dihydrate-Metformin co-crystals prepared according to the present disclosure when compared with the parent molecule was found to be greater than the parent molecule, i.e. Quercetin (a highly insoluble molecule).

Stability:

The stability of the Quercetin dihydrate-Metformin co-crystal was monitored, once in seven days for 3 months. The co-crystal were found to be stable under desiccated conditions, however, co-crystal tends to be hygroscopic under normal conditions.

In another embodiment, the disclosure describes a process for preparation of a pharmaceutical Quercetin dihydrate-antidiabetic co-crystal wherein said process comprises providing Quercetin with at least one of the antidiabetic agent; isolating said co-crystal and incorporating it into pharmaceutical composition along with one or more suitable pharmaceutical carriers/excipients. The Quercetin dihydrate-antidiabetic co-crystals are found to be more efficacious than the API, as seen from the Animal studies. Further, the pharmaceutical composition of the disclosure may be any pharmaceutical form which maintains the crystalline form of a co-crystal as disclosed herein.

The pharmaceutical composition may be a solid form, a liquid suspension or an injectable composition. The active ingredient (s) and excipients can be formulated into compositions and dosage forms according to methods known in the art.

The pharmaceutical composition of the disclosure may be prepared in the form of raw powders or granules dispersed in a suitable aqueous or non-aqueous liquid(s), pellets, beads, micro or nano particles, micro or a solvated powders, sachets, semisolids, an Injectable preparations, a tablets, a capsules or a suitable specific two- or three-dimensional matrix compositions. Preferably, the composition pharmaceutical composition is a single layer or a bilayer tablet.

The composition may be prepared by the techniques of dry granulation, wet granulation and/or direct compression using aqueous/Non aqueous solvent further fabricate into the single layer, bilayer, and multilayer and/or multicoated tablet dosage forms. The composition is prepared by the techniques of homogenization, emulsification comprising o/w, w/o or multiple emulsion (s), hot-melt fusion, lyophilization and/or precipitation.

The pharmaceutical composition of the disclosure may be extended to the development of micelle, emulsion and liposome formulation including small molecules, peptides, nuclei acids etc.

The disclosed co-crystal composition comprising Quercetin and another anti-diabetic agent is preferably administered along with at least one pharmaceutical excipient or carrier. The oral administration may be accomplished by ingesting the composition preferably in a form of tablet/capsule/liquid with a glass of water. The other dosage forms like hard gelatin capsules, powders, liquid capsules, syrups, suspensions, elixirs are also equally good modes of oral administration.

The quantity of the compound used in pharmaceutical co-crystal compositions as disclosed herein will vary depending upon the body weight of the patient and the mode of administration and can be of any effective amount to achieve the desired therapeutic effect.

In yet another embodiment, the disclosure describes a method for treating metabolic syndrome associated with Hypertension, diabetic condition inclusive of obesity and hyperlipidemia; which method comprises administering ‘an effective amount’ of the co-crystal composition comprising Quercetin and another anti-diabetic agent to the subject suffering from said disorder. The subject mentioned herein may be human. The composition may be administered as sustained release, controlled release, modified release, or immediate release dosage forms.

The ‘effective amount’ as described above means and includes the amount required to treat/alleviate the severity of symptoms associated with this ailments as decided by the persons of ordinary skill in the art.

In yet another embodiment, the current disclosure describes use of the co-crystals of quercetin and an antidiabetic agent in preparing a medicament intended to treat metabolic syndrome associated with Hypertension and diabetic condition inclusive of obesity and hyperlipidemia.

Anti-Hypertensive, Anti-Diabetic Activity Inclusive of Obesity and Hyperlipidemia:

The pharmaceutical co-crystals of Quercetin dihydrate-Metformin are tested for its anti-diabetic activity inclusive of obesity and hyperlipidemia as well as for hypertension by (i) determining the acute and chronic plasma glucose lowering activity of test compound in db/db mice and (ii) determining the chronic effect of test compounds on plasma insulin, triglyceride, cholesterol and body weight.

Methods:

Db/db mice were acclimatized to dosing and exposed to tail cut and r.o.p bleeding. Animals were grouped based on plasma glucose, triglyceride and cholesterol values. Acute effect of test compound on plasma glucose levels determined 3 hrs post dosing followed by administration of test compound for 21 days. After 14 days treatment, 4 hrs fasting plasma glucose levels were measured. After 21 days treatment, 4 hrs fasting plasma glucose levels were measured followed by test compound administration and measurement of plasma glucose levels 3 hrs post dosing.

Results:
Acute administration of ILB-MCO-0905Q and ILB-MCO-0906Q showed significant reduction in plasma glucose levels 3 hrs post dosing
After 21 days treatment test compounds did not show significant reduction in 4 hrs fasting plasma glucose levels. ILB-MCO-0905Q showed significant reduction in plasma insulin values compared to control group.
After 21 days treatment followed by acute administration of ILB-MCO-0905Q and ILB-MCO-0906Q, caused significant reduction in plasma glucose levels 3 hrs post dosing.

The results described herein above, conclusively proves the introduction of synergy in the co-crystals by combining the antidiabetics with the said co-crystal former, which act not only as co-crystal formers but also contribute to enhance the efficacy of the anti-diabetic agent by lowering the levels of plasma glucose, plasma insulin, triglyceride, cholesterol; improving the body weight and lowering the blood pressure, when compared to the administration of Quercetin and Metformin alone.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

### EXAMPLES

### Example 1

### Preparation of Metformin from Metformin Hydrochloride

1:1 molar ratio of Metformin hydrochloride and sodium hydroxide were dissolved in 2-Propanol. The suspension was stirred for 3 hours at 313K, filtered and the filtrate evaporated to yield a white solid free of chloride ion (checked with 0.1 M AgNO3 solution). The resultant product was confirmed as Metformin using IR, NMR studies and melting point (111° C.).

### Example 2

### Preparation of Metformin Free Base and Quercetin Dihydrate Co-Crystal

Quercetin dihydrate (heated to 150° C., 30.3 mg, and 0.1 mmol) and Metformin free base (25.8 mg, 0.2 mmol) were neat ground for 3 mins using a mortar and pestle. The resultant solid was subjected to analytical studies.

### Example 3

### Preparation of Quercetin Dihydrate and Metformin Hydrochloride Co-Crystal

Quercetin dihydrate (heated to 150° C., 30.3 mg, and 0.1 mmol) and Metformin hydrochloride (25.8 mg, 0.2 mmol) were neat ground for 3 mins using a mortar and pestle. The resultant solid was subjected to analytical studies.

Optionally, the co-crystals as disclosed herein can be prepared by melting method or solvent drop method using suitable solvents, followed by crystallization, if necessary.

The formation of these co-crystal or salt was confirmed by powder X-ray powder diffractometry and IR spectroscopy.

### Example 4

### Solubility Studies for Co-Crystals and Salts

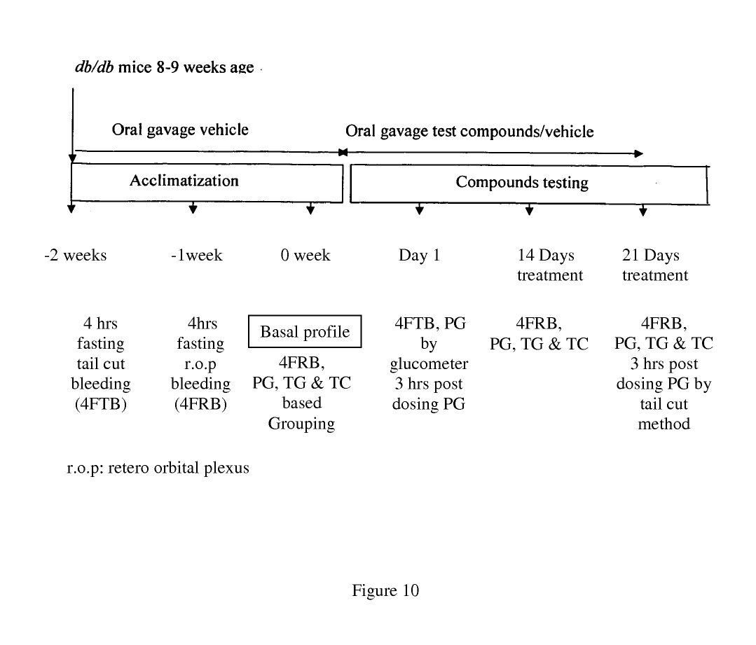

The solubility studies for co-crystals and salts were performed according to Higuchi and Connor's method with some variations. Excess amounts (100 mg) of the samples were suspended in 10 mL of water in round bottom flask. Solutions were stirred at 300 rpm using a magnetic stirrer. After 72 h, the suspensions were filtered through a paper filter (Whatman 40)) and filtered aliquots were sufficiently diluted, the absorbance of the samples were measured at 232 nm and the values were normalized for API. Finally, the concentration of API after 72 h (apparent aqueous solubility) in each sample was determined from the previously made standard graph. A standard graph was made by measuring the absorbance of varied concentrations of API in water using a UV spectrophotometer (Nanodrop UV/vis spectrometer) at λₘₐₓ 232 nm. (Refer FIGS. 10-12)

### Example 5

### Dissolution Profile of the Co-Crystals

Co-crystal are taken (2 mole) in 50 ml round bottom flask and 50 ml of nano pure water added over it. This solution was stirred at 300 rpm. 5 ml of solution was taken out at 5, 10, 30, 60 and 120 minutes interval and filtered through Whatman 40 filter paper. The filtrate was diluted by 10 times and UV absorption was measured.

### Example 6

### Clinical Study of the Co-Crystals

Anti-diabetic effect of ILB-MCO-0904Q (Quercetin dihydrate), ILB-MCO0905Q (Co-crystal of Quercetin dehydrate and Metformin Free base (1:2)) & ILB-MCO-0906Q (Metformin hydrochloride) in db/db mice:

Principle of Test:—

Primary Objective:

To determine the acute and chronic plasma glucose lowering activity of test compound in db/db mice.

Secondary Objective:

To determine the chronic effect of test compounds on plasma insulin, triglyceride, cholesterol and body weight.

Material and Methods:

Animals: In house colony db/db mice

Age: 7 weeks Sex: Male

Kits: Plasma glucose, cholesterol and triglyceride were measured using Dade Behring auto analyzer. Plasma insulin values measured using Rat/mouse insulin ELISA kit (Millipore, lot no 16006280). Tail cut method plasma glucose values measured using Contour TS glucometer strips (Lot WK8MD3E32A).

Compounds and Vehicle: All test-compounds were formulated in tween CMC (10% of 2.5% tween 80+90% of 0.25% CMC) and prepared daily about 1 hr before administration. Animals were dosed for 21 days through oral gavage at dose volume of 10 ml/kg body weight.

Groups:

Group 1: Vehicle control (n=10), 10 ml/kg, once daily, per oral

Group 2: Compound ILB-MCO-0904Q (n=10), 300 mg/kg, once daily, per oral

Group 3: Compound ILB-MCO-0905Q (n=10), 300 mg/kg, once daily, per oral

Group 4: Compound ILB-MCO-0906Q (n=10), 300 mg/kg, once daily, per oral

### Experimental Details

The experimental design for experiments on male db/db mice, summarized in FIG. 10, is described below. Male db/db mice, (bred in-house) aged around 7 weeks at the time of initiation of acclimatization, were used for the study. These mice, (5 per cage), were housed under standard temperature, light & humidity conditions and provided with NIN pellet feed and water, ad libitum.

During acclimatization period till the basal measurements, each mouse was orally dosed daily once with 0.4 ml of vehicle. At the end of first week of acclimatization animals were exposed to the stress of 4 hrs fasting, (feed removed at 7 AM.) and bleeding (at 11.00 AM morning), by tail cut method (side wise, small cut with scalpel blade 1 cm. above the tail end). After a gap of 2 days animals were exposed to stress of bleeding via retro-orbital plexus (r.o.p) after 4 hrs fasting. Basal plasma profile measurement (PG, TG & TC), in 4 hrs fasted mice, was conducted at the end of 2nd week of acclimatization. Animals were grouped based on basal plasma glucose, triglyceride levels and cholesterol levels. Plasma samples were frozen for insulin measurement.

Acute Effect Measurement:

On the day of initiating study animals were fasted for 4 hrs and plasma glucose was measured with Glucometer by tail cut method. Animals were dosed with test compound/vehicle according to their body weight and 3 hrs post dosing plasma glucose levels were measured with Glucometer Immediately animals were provided with measured amount of feed.

Chronic Effect:

Animals were dosed daily at around 10 AM to 11 AM with corresponding test compounds/vehicle at a volume of 10 ml/kg of body weight. Body weight, water intake and food intake was measured daily. After 14 days treatment plasma profile (PG, TG & TC) was measured in 4 hrs fasted animals. On that day animals were dosed post bleeding after keeping feed at 4 PM. After 21 days treatment plasma profile (PG, TG & TC) was measured in 4 hrs fasted animals. After bleeding, animals were administered with test compounds/vehicle and plasma glucose was measured 3 hrs post dosing with Glucometer. Immediately animals were provided with feed.

Blood samples from r.o.p bleeding were collected in micro centrifuge tubes containing 5 ul of EDTA Na (200 ng/ml). Plasma separated by centrifuging blood samples at 6000 rpm at 40 C for 5 minutes. Plasma glucose, total cholesterol and triglyceride were measured using Dade Bhering auto analyser. Plasma insulin was measured (in duplicate samples), using Millipore mouse/rat insulin ELISA kit.

Calculation & Statistics:

Acute effect percent reduction in PG was calculated for each animal using the formula

{(0 hr PG−test hr PG)/0 hr PG}×100

The percent reduction in plasma profile parameters were calculated using the formula 1−(tt/tc)/bt/bc)×100; where tt: test day treated group mean value of the parameter; tc: test day control group mean value of the parameter; bc: Basal Control group mean value; bt: Basal treated group mean value.

Statistics were applied using One-Way ANOVA followed by Dunnett's test using sigma stat software. The values shown in the tables are Mean±SEM

Results:

Acute Effect:

TABLE 1
Acute effect of test compounds on plasma glucose
levels in d/b/db mice (mg/dl; Mean ± SEM)

| | | | % |
|---|---|---|---|
| Group | 0 Hr | 3 Hr | Reduction |
| Vehicle Control | 377 ± 44 | 290 ± 39 | 23 ± 4 |
| (n = 10) | | | |
| ILB-MCO-0904Q | 398 ± 67 | 293 ± 65 | 31 ± 4 |
| 300 mg/kg (n = 10) | | | |
| ILB-MCO-0905Q | 358 ± 34 | 186 ± 21 | 48 ± 4* |
| 300 mg/kg (n = 10) | | | |
| ILB-MCO-0906Q | 373 ± 43 | 181 ± 25 | 52 ± 4* |
| 300 mg/kg (n = 10) | | | |

*P < 0.05 vs control group

Chronic Effect: Post 4 Hr Fasting, Pre Dosing, 14 Days

TABLE 2
Chronic effect of test compounds on plasma glucose levels (mg/dl; Mean ± SEM) before dosing on the day of bleeding

| | Basal | 14th day | % | | 14th day | % | | 14th day | % |
|---|---|---|---|---|---|---|---|---|---|
| Group | PG | PG | Red | Basal TC | TC | Red | Basal TG | TG | Red |
| Vehicle | 395.36 ± 31.93 | 449.24 ± 37.50 | — | 155.90 ± 5.03 | 153.02 ± 4.41 | — | 146.70 ± 8.49 | 149.08 ± 11.31 | — |
| Control | | | | | | | | | |
| (n = 10) | | | | | | | | | |
| ILB-MCO- | 402.50 ± 26.82 | 476.41 ± 45.57 | −4 | 156.25 ± 4.53 | 154.43 ± 6.50 | −1 | 138.00 ± 8.04 | 129.08 ± 5.61 | 8 |
| 0904Q | | | | | | | | | |
| 300 mg/kg | | | | | | | | | |
| (n = 10) | | | | | | | | | |
| ILB-MCO- | 399.40 ± 26.54 | 417.71 ± 37.85 | 8 | 160.42 ± 3.77 | 153.68 ± 3.49 | 2 | 135.94 ± 6.92 | 126.70 ± 6.25 | 8 |
| 0905Q | | | | | | | | | |
| 300 mg/kg | | | | | | | | | |
| (n = 10) | | | | | | | | | |
| ILB-MCO- | 402.43 ± 26.13 | 443.33 ± 40.27 | 3 | 155.90 ± 5.08 | 144.51 ± 4.61 | 6 | 136.10 ± 5.39 | 114.11 ± 4.76 | 17 |
| 0906Q | | | | | | | | | |
| 300 mg/kg | | | | | | | | | |
| (n = 10) | | | | | | | | | |

Chronic Effect: Post 4 Hr Fasting, Predosing, 21 Days

TABLE 3
Chronic effect of test compounds on plasma glucose levels (mg/dl; Mean ± SEM) before dosing on the day of bleeding

| | Basal | 21st day | % | | 21st day | % | | 21st day | % |
|---|---|---|---|---|---|---|---|---|---|
| Group | PG | PG | Red | Basal TC | TC | Red | Basal TG | TG | Red |
| Vehicle | 395.36 ± 31.93 | 425.01 ± 42.71 | — | 155.90 ± 5.03 | 178.20 ± 24.04 | — | 146.70 ± 8.49 | 190.52 ± 25.66 | — |
| Control | | | | | | | | | |
| (n = 9) | | | | | | | | | |
| ILB- | 402.50 ± 26.82 | 432.69 ± 42.38 | 0 | 156.25 ± 4.53 | 151.25 ± 4.93 | 15 | 138.00 ± 8.04 | 141.99 ± 8.25 | 21 |
| MCO- | | | | | | | | | |
| 0904Q | | | | | | | | | |
| 300 mg/kg | | | | | | | | | |
| (n = 10) | | | | | | | | | |
| ILB- | 399.40 ± 26.54 | 368.96 ± 32.95 | 14 | 160.42 ± 3.77 | 160.25 ± 4.29 | 13 | 135.94 ± 6.92 | 129.66 ± 5.38 | 27 |
| MCO- | | | | | | | | | |
| 0905Q | | | | | | | | | |
| 300 mg/kg | | | | | | | | | |
| (n = 10) | | | | | | | | | |
| ILB- | 402.43 ± 26.13 | 400.80 ± 42.77 | 7 | 155.90 ± 5.08 | 157.86 ± 8.76 | 11 | 136.10 ± 5.39 | 127.00 ± 5.65 | 28 |
| MCO- | | | | | | | | | |
| 0906Q | | | | | | | | | |
| 300 mg/kg | | | | | | | | | |
| (n = 10) | | | | | | | | | |

Chronic Effect: 3 Hr Post Dosing, 21 Days

TABLE 4
Chronic effect (21 days) of test compounds on plasma
glucose 3 hrs post dosing (mg/dl; Mean ± SEM)

| | 21th day | |
|---|---|---|
| | 3 hr post dosing | % |
| Group | PG | Red |
| Vehicle Control | 360 ± 47 | — |
| (n = 9) | | |
| ILB-MCO-0904Q | 345 ± 41 | 4 |
| 300 mg/kg (n = 10) | | |
| ILB-MCO-0905Q | 225 ± 33* | 38 |
| 300 mg/kg (n = 10) | | |
| ILB-MCO-0906Q | 200 ± 31* | 44 |
| 300 mg/kg (n = 10) | | |

*P < 0.05 vs control group

Chronic Effect: Post 4 Hr Fasting, Predosing, 21 Days Treatment

TABLE 5
Chronic effect of test compounds on plasma insulin levels (ng/ml; Mean ± SEM)

| | Basal | 21th day | % | % |
|---|---|---|---|---|
| Group | insulin | insulin | Reduction | Reduction@ |
| Vehicle Control | 3.90 ± 0.38 | 4.69 ± 0.77 | −21.81 ± 19.11 | — |
| (n = 9) | | | | |
| ILB-MCO-0904Q | 2.94 ± 0.20 | 2.90 ± 0.33 | 0.29 ± 9.87 | 18 |
| 300 mg/kg (n = 10) | | | | |
| ILB-MCO-0905Q | 4.55 ± 0.50 | 3.03 ± 0.42 | 30.56 ± 8.10* | 45 |
| 300 mg/kg (n = 10) | | | | |
| ILB-MCO-0906Q | 4.42 ± 0.63 | 3.82 ± 0.72 | 15.32 ± 7.16 | 28 |
| 300 mg/kg (n = 10) | | | | |

*P < 0.05 vs control group
@calculated after adjusting for control group change

TABLE 6
Chronic effect of test compounds
on body weight (mg; Mean ± SEM)

| | Basal | Final day | % |
|---|---|---|---|
| Group | Body weight | Body weight | Increase |
| Vehicle Control | 39.1 ± 1.2 | 44.1 ± 1.1 | 11.2 ± 2.3 |
| (n = 9) | | | |
| ILB-MCO-0904Q | 39.1 ± 1.2 | 44.6 ± 1.3 | 12.2 ± 2.3 |
| 300 mg/kg (n = 10) | | | |
| ILB-MCO-0905Q | 39.2 ± 0.8 | 43.7 ± 1.0 | 10.1 ± 2.5 |
| 300 mg/kg (n = 10) | | | |
| ILB-MCO-0906Q | 38.5 ± 0.5 | 45.1 ± 0.6 | 14.5 ± 0.9 |
| 300 mg/kg (n = 10) | | | |

Summary of Test Compound Effects in Db/Db Mice:
ILB-MCO-0904Q 300 mg/kg: Did not show significant effect on plamsa glucose levels with acute or chronic administration.
ILB-MCO-0905Q 300 mg/kg: Significant lowering of plasma glucose levels 3 hrs post dosing with single dose. Chronic treatment (21 days) did not show significant reduction in plasma glucose levels but showed significant reduction in plasma insulin levels. This, followed by compound administration on day 21, showed significant lowering of plasma glucose levels.
ILB-MCO-0906Q 300 mg/kg: Significant lowering of plasma glucose levels 3 hrs post dosing with single dose. Chronic treatment (21 days) did not show significant reduction in plasma glucose levels. This, followed by compound administration on day 21, showed significant lowering of plasma glucose levels.
ILB-MCO-0904Q, ILB-MCO-0905Q and ILB-MCO-0906Q showed 15%, 13% and 11% reduction in plasma cholesterol levels respectively, after 21 days treatment post 4 hrs fasting, without dosing on the day.
ILB-MCO-0904Q, ILB-MCO-0905Q and ILB-MCO-0906Q showed 21%, 27% and 28% reduction in plasma triglyceride levels respectively, after 21 days treatment post 4 hrs fasting, without dosing on the day.
Animal number 5 belongs to control group was found dead on 3 Aug. 2009 and was examined by necropsy. This animal lost around 4.5 g body weight from 30 Jun. 2009 to 3 Aug. 2009. The exact cause of animal death could not be ascertained. However, there were no significant changes in body weight with test compounds administration compared to control group.

We claim:

1. A co-crystal composition comprising:
a) Quercetin, a hydrate thereof, a polymorph thereof, a salt thereof, or mixtures thereof; and
b) at least one anti-diabetic agent selected from the group consisting of DPP-IV inhibitors.

2. The co-crystal composition as claimed in claim 1, wherein the DPP-IV inhibitors are selected from the group consisting of sitagliptin, vildagliptin, dutogliptin, saxagliptin, linagliptin, gemigliptin, alogliptin and mixtures thereof.

3. The co-crystal composition as claimed in claim 1, wherein said co-crystal composition comprises a hydrate of quercetin or a polymorph of quercetin.

4. A composition comprising:
a) a co-crystal comprising:
i) Quercetin, a hydrate thereof, a polymorph thereof, a salt thereof, or mixtures thereof; and
ii) at least one first anti-diabetic agent; and
b) a second anti-diabetic agent selected from the group consisting of biguanide drugs; sulfonylurea drugs; alpha-glucosidase inhibitors; thiazolidinedione drugs; DPP-IV inhibitors, and mixtures thereof.

5. The co-crystal composition as claimed in claim 4, wherein the co-crystal is in physical combination with said second anti-diabetic agent.

6. The co-crystal composition as claimed in claim 5, wherein said second anti-diabetic agent is selected from the group consisting of tolazamide, glipizide, glimepiride, acarbose, rosiglitazone, pioglitazone, miglitol, sitagliptin, vildagliptin, dutogliptin, saxagliptin, linagliptin, gemigliptin, alogliptin, and mixtures thereof.

7. The co-crystal composition as claimed in claim 5, wherein said first anti-diabetic agent is selected from the group consisting of metformin, salts thereof, polymorphs thereof, hydrates thereof, and mixtures thereof.

8. The co-crystal composition as claimed in claim 4, wherein:
said first anti-diabetic agent is selected from the group consisting of metformin, salts thereof, polymorphs thereof, hydrates thereof, and mixtures thereof; and
said second anti-diabetic agent is selected from the group consisting of DPP-IV inhibitors.

9. The co-crystal composition as claimed in claim 1, wherein said composition further comprises one or more suitable pharmaceutically acceptable diluents, carriers or excipients.

10. The composition as claimed in claim 4, wherein said composition further comprises one or more suitable pharmaceutically acceptable diluents, carriers or excipients.

11. An oral dosage form comprising a co-crystal composition as claimed in claim 1, wherein said oral dosage form is selected from the group consisting of tablets, powders, capsules, syrups, suspensions, elixirs, a single layer tablet, a bilayer tablet, and liquid dosage forms.

12. A parenteral dosage form comprising a co-crystal composition as claimed in claim 1.

13. An oral dosage form comprising a composition as claimed in claim 4, wherein said oral dosage form is selected from the group consisting of tablets, powders, capsules, syrups, suspensions, elixirs, a single layer tablet, a bilayer tablet, and liquid dosage forms.

14. A parenteral dosage form comprising a composition as claimed in claim 4.

15. An oral dosage form comprising a co-crystal composition as claimed in claim 1, wherein said oral dosage form is selected from the group consisting of sustained release, controlled release, modified release and immediate release dosage forms.

16. An oral dosage form comprising a composition as claimed in claim 4, wherein said oral dosage form is selected from the group consisting of sustained release, controlled release, modified release and immediate release dosage forms.

17. A dosage form comprising a co-crystal composition as claimed in claim 1, wherein said pharmaceutical co-crystal composition is in the form of a powders or granules dispersed in a carrier.

18. A dosage form as claimed in claim 17, wherein said carrier is selected from the group consisting of an aqueous liquid, a non-aqueous liquid, pellets, beads, micro- or nanoparticles, a powder, a micropowders, sachets, a semisolid matrix, a tablet, a capsule, and a two- or three-dimensional matrix composition.

19. A dosage form comprising a composition as claimed in claim 4, wherein said pharmaceutical co-crystal composition is in the form of a powders or granules dispersed in a carrier.

20. A dosage form as claimed in claim 19, wherein said carrier is selected from the group consisting of an aqueous liquid, a non-aqueous liquid, pellets, beads, micro- or nanoparticles, a powder, a micropowders, sachets, a semisolid matrix, a tablet, a capsule, and a two- or three-dimensional matrix composition.

21. A dosage form prepared from a co-crystal composition as claimed in claim 1, wherein:
said co-crystal composition is shaped by at least one process selected from the group consisting of dry granulation, wet granulation and direct compression to form an intermediate product;
wherein an aqueous or non-aqueous solvent is optionally used in said shaping process; and
wherein said intermediate product is optionally fabricated into a dosage form selected from the group consisting of a single layer tablet, a bilayer tablet, a multilayer tablet and a tablet having at least one coating layer.

22. A dosage form prepared from a co-crystal composition as claimed in claim 1, wherein:
said dosage form is prepared by homogenization, oil-in-water emulsification, water-in-oil emulsification, multiple emusification, hot-melt fusion, lyophilization or precipitation.

23. A method for treatment of a disease selected from the group consisting of diabetic cataract, metabolic syndrome, hypertension, diabetes, obesity and hyperlipidemia, comprising:
administering an effective amount of a co-crystal composition according to claim 1 to a subject suffering from said disease.

24. A method for treatment of a disease selected from the group consisting of diabetic cataract, metabolic syndrome, hypertension, diabetes, obesity and hyperlipidemia, comprising:
administering an effective amount of a composition according to claim 4 to a subject suffering from said disease.

25. The method of claim 23, wherein said subject is a human subject.

26. The method of claim 24, wherein said subject is a human subject.

27. A pharmaceutical composition effective for treatment of diabetic cataract, comprising an effective amount of a co-crystal composition;
said co-crystal composition comprising:
a) Quercetin, a hydrate thereof, a polymorph thereof, a salt thereof, or mixtures thereof; and
b) at least one first anti-diabetic agent.

28. The pharmaceutical composition of claim 27, wherein said composition is an oral dosage form selected from the group consisting of single-layer tablets, bilayer tablets, multilayer tablets, powders, capsules, syrups, suspensions, elixirs, liquid dosage forms, sustained release dosage forms, controlled release dosage forms, modified release dosage forms, and immediate release dosage forms.

29. The pharmaceutical composition of claim 27, wherein said co-crystal composition is in physical combination with a second anti-diabetic agent selected from the group consisting of sulfonylurea compounds, alpha-glucosidase inhibitors, thiazolidinedione compounds, DPP-IV inhibitors, and mixtures thereof.

30. The pharmaceutical composition of claim 27, wherein said composition is a parenteral dosage form.

31. The pharmaceutical composition of claim 27, wherein said at least one antidiabetic agent is selected from the group consisting of metformin, salts thereof, polymorphs thereof, hydrates thereof, and mixtures thereof.

32. The pharmaceutical composition of claim 27, wherein said at least one antidiabetic agent is selected from the group consisting of DPP-IV inhibitors.

33. A method of treating diabetic cataract in a patient, comprising administering to said patient an effective amount of a co-crystal composition according to claim 27.
